(19) United States
(12) Patent Application Publication
(10) Pub. No.: US 2019/0269942 A1
(43) Pub. Date: Sep. 5, 2019

(54) ULTRASOUND THERAPY FOR BLADDER DYSFUNCTION

(71) Applicant: Medtronic, Inc., Minneapolis, MN (US)

(72) Inventors: Jamu K. Alford, Simi Valley, CA (US); Yohan Kim, Fridley, MN (US); John R. LaLonde, Lake Elmo, MN (US); Xuan K. Wei, Minnetonka, MN (US)

(21) Appl. No.: 16/291,764

(22) Filed: Mar. 4, 2019

Related U.S. Application Data
(60) Provisional application No. 62/638,634, filed on Mar. 5, 2018.

(51) Int. Cl. A61N 7/00; A61B 8/08; A61B 5/20; A61B 5/0205; A61N 7/02; A61B 5/00

(52) U.S. Cl. CPC A61N 7/00; A61B 8/08; A61B 5/204; A61B 5/0205; A61B 5/024; A61B 5/4836; A61N 2007/0026; A61N 2007/0073; A61N 7/02

(57) ABSTRACT

The disclosure describes devices, systems, and techniques for delivering therapy configured to treat bladder dysfunction. For example, a system may be configured to modulate bladder activity by determining a set of parameter values that at least partially defines ultrasound energy configured to modulate nerve tissue associated with bladder activity of a patient. The system may then control, according to the set of parameter values, delivery of the ultrasound energy from a set of ultrasound transducers external to the patient and to the nerve tissue associated with the bladder of the patient to modulate bladder activity and influence patient voiding.

This application claims the benefit of U.S. Provisional Application Ser. No. 62/638,634 entitled “ULTRASOUND THERAPY FOR BLADDER DYSFUNCTION” and filed Mar. 5, 2018, the entire contents of which are incorporated herein by reference.

### TECHNICAL FIELD

The disclosure relates to medical therapies and, more particularly, delivery of ultrasound therapy.

### BACKGROUND

Delivery of ultrasound involves delivering sound waves with frequencies higher than the upper audible limit of human hearing. Delivery of ultrasound is performed for diagnostic imaging, e.g., to visualize internal body structures such as tendons, muscles, joints, vessels, and internal organs. Ultrasound images are made delivering ultrasound, e.g., pulses of ultrasound, into tissue using one or more ultrasound transducers. The sound echoes, or reflects, off the tissue, with different tissues having different characteristics reflecting and diffracting the sound differently. The reflected and diffracted sound is sensed by one or more ultrasound transducers.

Ultrasound has also been delivered to patients for therapeutic purposes. For example, ultrasound has been delivered to promote healing and/or blood flow. As another example, ultrasound has been delivered to modify or destroy problematic tissue, such as tumors. Delivery of ultrasound for medical purposes often involves a relatively-large, cart-based piece of equipment that includes, for example, circuitry for generating and sensing ultrasound signals, processing circuitry, a user interface, and an internal power source and/or the ability to be plugged to AC mains power. A probe may be hand-held and include the one or more ultrasound transducers connected to ultrasound device by a cable.

### SUMMARY

In general, the disclosure is directed to techniques and/or systems for delivering therapy configured to treat bladder dysfunction of a patient. For example, a system may deliver ultrasound energy to nerve tissue associated with activity of the bladder, such as stretch receptors on the bladder and/or efferent nerves that contribute to bladder over-activity. The ultrasound transducers may be part of an array of ultrasound transducers part of a patch or other wearable device attached to the patient. The system may select appropriate transducers for targeting desired nerve tissue within the patient and/or select transducers to image patient anatomy and/or physiological events such as bladder and/or nerve activity. In some examples, the system may trigger the start of ultrasound therapy delivery in response to detecting an event via the ultrasound transducers and/or via another type of sensor configured to monitor the patient (e.g., an accelerometer, a set of electrodes, or a temperature sensor).

In one example, a method for modulating bladder activity includes determining, by processing circuitry, a set of parameter values that at least partially defines ultrasound energy configured to modulate nerve tissue associated with activity of a bladder of a patient, and controlling, by processing circuitry and according to the set of parameter values, delivery of the ultrasound energy from a set of ultrasound transducers external to the patient and to the nerve tissue associated with the activity of the bladder of the patient.

In another example, a system for modulating bladder activity includes processing circuitry configured to determine a set of parameter values that at least partially defines ultrasound energy configured to modulate nerve tissue associated with activity of a bladder of a patient, and control, according to the set of parameter values, delivery of the ultrasound energy from a set of ultrasound transducers external to the patient and to the nerve tissue associated with the activity of the bladder of the patient.

In another example, a non-transitory computer readable medium includes instructions that, when executed, cause processing circuitry to determine a set of parameter values that at least partially defines ultrasound energy configured to modulate nerve tissue associated with activity of a bladder of a patient, and control, according to the set of parameter values, delivery of the ultrasound energy from a set of ultrasound transducers external to the patient and to the nerve tissue associated with the activity of the bladder of the patient.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a conceptual diagram illustrating an example system for delivering ultrasound therapy configured to treat bladder dysfunction of a patient via a wearable ultrasound device.

FIG. 1B is a conceptual diagram illustrating another example system for delivering ultrasound therapy configured to treat bladder dysfunction of a patient via a wearable ultrasound device.

FIGS. 2A and 2B are top-view and side-view diagrams, respectively, illustrating an example wearable ultrasound device.

FIG. 3 is a functional block diagram illustrating an example configuration of a wearable ultrasound device.

FIG. 4 is a functional block diagram illustrating an example configuration of an interface device.

FIG. 5 is a flow diagram illustrating an example technique of modulating bladder activity of a patient via a wearable ultrasound device.

FIG. 6 is a flow diagram illustrating an example technique of determining a set of parameters based on imaging via ultrasound transducers of a wearable ultrasound device.

FIG. 7 is a flow diagram illustrating an example technique of delivering ultrasound energy to nerve tissue of a patient based on detection of a physiological event.

### DETAILED DESCRIPTION

The disclosure is directed to techniques, systems, and devices configured to deliver ultrasound therapy configured to treat pelvic dysfunctions that may include urinary or bladder dysfunction and/or fecal dysfunction of a patient. Bladder dysfunction generally refers to a condition of improper functioning of the bladder or urinary tract, and may include, for example, an overactive bladder, urgency, and urinary incontinence. Urgency is a sudden, compelling urge to urinate, and may often, though not always, be associated with urinary incontinence. Overactive bladder may include excessive contractions of the detrusor muscle (e.g., smooth muscle residing in the wall of the bladder) and may be one of the causes for urgency. Urinary incontinence refers to a condition of involuntary loss of urine, and may include urge incontinence, stress incontinence, or both stress and urge incontinence, which may be referred to as mixed urinary incontinence. As used in this disclosure, the term “urinary incontinence” includes disorders in which urination occurs when not desired, such as stress or urge incontinence (e.g., overactive bladder).

In order to void urine, the nervous system and several muscles of the body typically work in concert to expel urine from the bladder. For example, the internal urinary sphincter muscle and the external urinary sphincter muscle relax to allow urine to pass through the openings in these sphincters. In addition, the detrusor muscle in the wall of the bladder contracts to increase the internal bladder pressure and force urine out of the bladder and through the urethra and past the urinary sphincters. Bladder dysfunction can occur when portions of the nervous system that innervate these muscles, or the muscles themselves, prevent the patient from retaining urine until the patient voluntarily decides to urinate.

For example, urge incontinence may be caused by disorders of peripheral or central nervous systems that control bladder micturition reflexes. Some patients may also suffer from nerve disorders that prevent proper triggering and operation of the bladder, sphincter muscles or nerve disorders that lead to overactive bladder activities or urge incontinence. Additionally, or alternatively, urinary incontinence can be attributed to improper sphincter function, either in the internal urinary sphincter or external urinary sphincter. In some examples, aging can result in weakened sphincter muscles, which may cause incontinence. Nerves running though the pelvic floor stimulate contractility in the sphincter. An improper communication between the nervous system and the urethra or urinary sphincter can result in a bladder dysfunction, such as overactive bladder, urgency, urge incontinence, or another type of urinary incontinence.

Some treatments for bladder disorders such overactive bladder or urge incontinence may include pharmaceutical treatment from one or more drugs intended to relax the detrusor muscle and increase the amount of urine held by the patient. In other examples, electrical stimulation therapy may be employed when pharmaceutical treatment is not effective. In electrical stimulation therapy, one or more medical leads carrying electrodes may be implanted into the patient and coupled to an implanted medical device (IMD). The IMD may then generate electrical stimulation pulses and deliver those pulses to nerves or muscles via the electrodes to reduce detrusor muscle activity and/or increase sphincter contraction, as necessary. However, electrical stimulation therapy requires the surgical implantation of devices into the patient. Implantation of a stimulation device may involve one or more time-consuming medical procedures, increased patient recovery time, increased cost, or the like.

As described in this disclosure, various techniques and systems may be used to modulate bladder activity to treat bladder dysfunction of a patient. For example, a system may include ultrasound transducers configured to deliver ultrasound energy to one or more nerve tissues of the patient to inhibit or activate various muscles. The system may determine a set of parameter values at least partially defining ultrasound energy, and control ultrasound energy delivered to nerve tissue associated with activity of a bladder of the patient. The nerve tissue may innervate the bladder or other muscles associated with bladder function, such as the urinary or external sphincters. The system may control a set of ultrasound transducers external to the patient to deliver the ultrasound energy to target nerve tissue associated with bladder activity. In this way, the ultrasound therapy configured to treat bladder dysfunction of the patient as described herein may be less invasive than some other therapy delivery methods, such as electrical stimulation therapy delivered from implanted devices.

In some examples, placement of such invasive stimulation devices near desired nerve tissue configured to innervate the bladder of the patient may present additional challenges. For example, placement of an electrical lead near nerve tissue configured to innervate the bladder that is relatively far from the bladder itself may result in stimulation of nerve tissue configured to innervate other organs in addition to the bladder. In turn, one or more additional organs may be stimulated in response to the stimulation intended to innervate only the bladder or related structures. Moreover, placement of an electrical lead near nerve tissue relatively close to the bladder itself may be more difficult to identify, more difficult to position the electrical lead near, or smaller than nerve tissue farther from the bladder, all of which may lead to less effective outcomes. In this way, the medical procedure to implant the electrical lead may be more difficult to achieve desired therapeutic outcomes, more time-consuming, or both. The use of the techniques and systems described herein to non-invasively modulate bladder activity to treat bladder dysfunction of a patient may reduce the number of invasive medical procedures experienced by the patient, patient recovery time, clinician time, cost, or the like.

Although certain aspects and examples of this disclosure are generally directed to treating urinary and/or bladder dysfunction, such as dysfunction related to holding and releasing urine, this disclosure is not meant to be limiting to urinary and/or bladder dysfunction. For example, systems and techniques described herein may instead, or additionally, be used to treat fecal incontinence or dysfunction related to the rectum and/or bowel. For example, a system may control the delivery of ultrasound energy to nerves innervating the bowel and/or anal sphincters to improve the retention of fecal matter for the patient. In this manner, examples herein are directed toward pelvic dysfunctions that may include urinary dysfunction and/or fecal dysfunction.

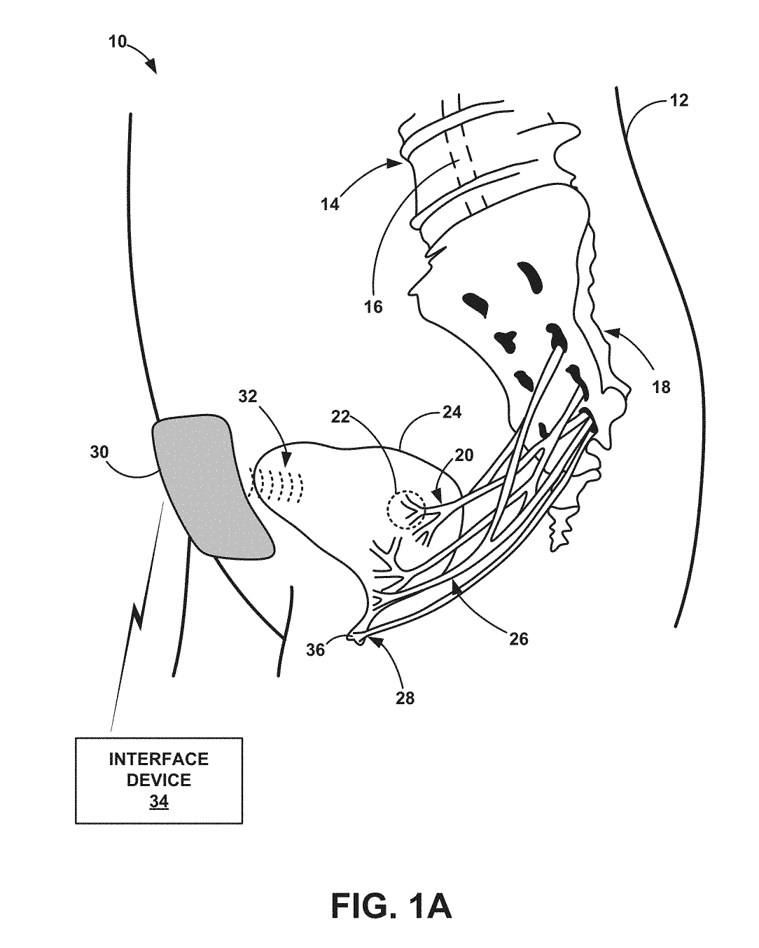

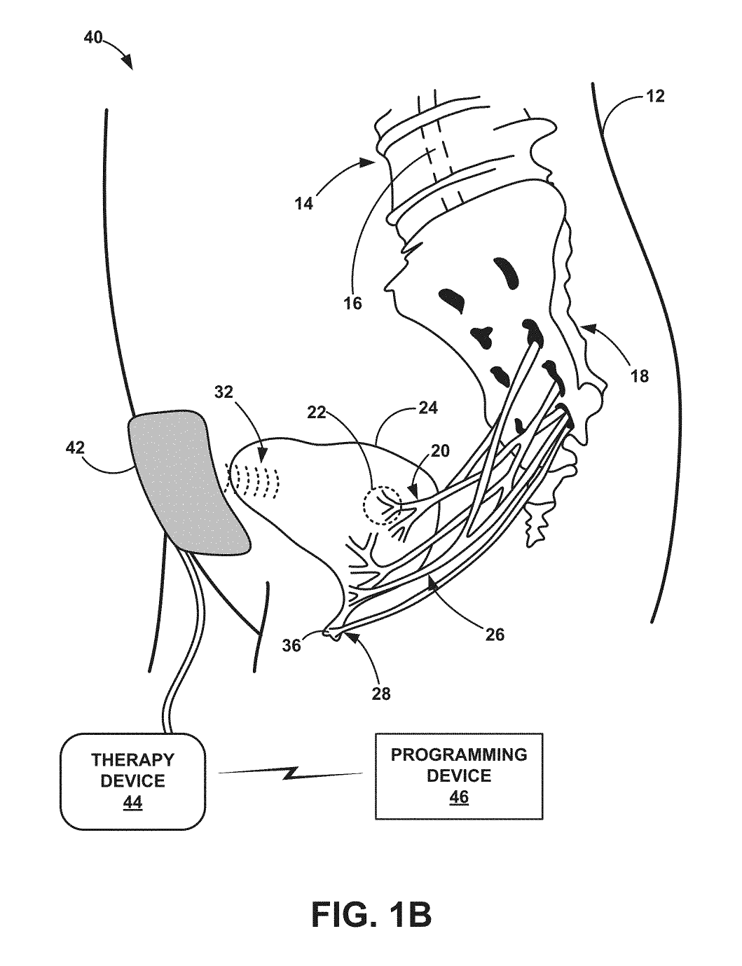

FIG. 1A is a conceptual and schematic diagram illustrating an example system 10 for delivering therapy configured to treat bladder dysfunction of a patient 12 via a wearable ultrasound device 30. Patient 12 ordinarily will be a human patient. In some cases, however, system 10 may be applied to other mammalian or non-mammalian, non-human patients. In some examples, patient 12 experiences bladder dysfunction, such as, for example, improper functioning of a bladder 24, a urinary sphincter 36, or a urinary tract, and may include an overactive bladder, urgency, urinary incontinence, or combinations thereof.

As will be described in greater detail below, wearable ultrasound device 30 may include a plurality of ultrasound transducers, signal generation circuitry configured to drive the plurality of ultrasound transducers, one or more power sources configured to power the signal generation circuitry, and one or more processors or other processing circuitry configured to control the signal generation circuitry. The plurality of ultrasound transducers of wearable ultrasound device 30 are configured to deliver ultrasound energy 32 to nerve tissue associated with activity of bladder 24, such as nerve tissue that at least partially innervates bladder 24 of patient 12. In some examples, the plurality of ultrasound transducers may include one or more sets of ultrasound transducers. As used in this disclosure, the phrase “set of ultrasound transducers” may refer to a single ultrasound transducer or more than one ultrasound transducer. For example, a set of ultrasound transducers may include 1 ultrasound transducer, 2 ultrasound transducers, 3 ultrasound transducers, 4 ultrasound transducers, or more than 4 ultrasound transducers. In some examples, the set of ultrasound transducers may include tens, hundreds, or even thousands of ultrasound transducers.

Wearable ultrasound device 30 is configured to deliver ultrasound energy 32 from the plurality of ultrasound transducers external to nerve tissue associated with bladder 24 of patient 12. In this way, system 10 provides a non-invasive way to modulate activity of bladder 24 and/or associated structures. In some examples, wearable ultrasound device 30 may include an attachment element, such as an adhesive layer, for attaching the device to patient 12. In addition to, or instead of an adhesive layer, in some examples, an attachment element may include a strap or garment. In turn, patient 12 may be able to position and maintain the position of the ultrasound transducers of wearable ultrasound device 30 relative to a treatment or diagnostic area of patient 12. For example, wearable ultrasound device 30 may be an external patch configured to be attached to an external skin surface of patient 12.

Wearable ultrasound device 30 may be positioned at one or more external positions of patient 12. In some examples, the position of wearable ultrasound device 30 may be selected based on the nerve tissue wearable ultrasound device 30 is configured to modulate. For example, wearable ultrasound device 30 may be positioned at a midline of a suprapubic area of patient 12 (e.g., to be positioned approximately near bladder 24) in cases in which wearable ultrasound device 30 is configured to deliver ultrasound energy 32 to nerve tissue 26 (efferent nerves such as sympathetic and/or parasympathetic nerves), afferent nerves 20, and/or stretch receptors 22. In other cases, such as if wearable ultrasound device 30 is configured to deliver ultrasound energy 32 to pudendal nerve 28, wearable ultrasound device 30 may also be positioned in a suprapubic area, but other locations are also contemplated. For example, wearable ultrasound device 30 may be positioned at the perineum of patient 12. As yet another example, wearable ultrasound device 30 may be positioned at or near the genitals of patient 12, such as if wearable ultrasound device 30 is configured to deliver ultrasound energy 32 to a dorsal genital nerve. As still another example, wearable ultrasound device 30 may be configured to deliver ultrasound energy 32 to a sacral dermatome and/or a sacral nerve, and may be positioned on the lower back or upper buttocks area of patient 12. In other examples, wearable ultrasound device 30 may be positioned differently based on the nerve tissue wearable ultrasound device 30 is configured to modulate, or wearable ultrasound device 30 may not be positioned based on the nerve tissue wearable ultrasound device 30 is configured to modulate. Since wearable ultrasound device 30 may be configured to direct ultrasound to numerous locations from an array of ultrasound transducers, exact location of wearable ultrasound device may not be crucial to the ability of the device to target certain nerve tissues targeted for treatment.

In some examples, the components of wearable ultrasound device 30 may be configured, e.g., constructed and arranged, such that wearable ultrasound device 30 is flexible. In some examples, wearable ultrasound device 30 is flexible such that it conforms to a surface of patient 12 on which the wearable ultrasound device is attached. Wearable ultrasound device 30 may be used, and attached to patient 12, for time periods as brief as a few minutes to as long as several months. The flexibility of wearable ultrasound device 30 may increase the comfort of patient 12.

Wearable ultrasound device 30 includes one or more power sources. The one or more power sources may include an attached battery, such as an attached rechargeable battery or an attached primary battery. In examples in which wearable ultrasound device 30 includes a rechargeable battery, wearable ultrasound device 30 may include a recharge interface, such as a coil for inductive recharging or connector, e.g., universal serial bus (USB), mini-USB, or micro-USB, for wired recharging of the one or more power sources. In some examples, an interface device 34 or another device may charge the one or more power sources of wearable ultrasound device 30.

Ultrasound energy 32 may be delivered to patient 12 by a set of ultrasound transducers for therapeutic purposes, such as to modulate activity of bladder 24. For example, ultrasound energy 32 may modulate nerve tissue that extends from sacrum 18 that at least partially innervates bladder 24. In some examples, ultrasound energy 32 modulates nerve tissue 26 including one or more pelvic nerves (e.g., parasympathetic nerves) and/or hypogastric nerves (e.g., sympathetic nerves). In some such examples, ultrasound energy 32 may modulate nerve tissue 26 to stimulate or inhibit the relaxation and/or contraction of the detrusor muscle (e.g., smooth muscle of the wall of bladder 24) to treat bladder dysfunction of patient 12.

Additionally, or alternatively, ultrasound energy 32 may be configured to modulate nerve tissue including afferent nerves 20 that at least partially innervates bladder 24 of patient 12. Afferent nerves 20 are sensory nerves that terminate in stretch receptors 22 on bladder 24. Ultrasound energy 32 may modulate afferent nerves 20 and/or stretch receptors 22 to stimulate or inhibit signals from afferent nerves 20 and/or stretch receptors 22 to the brain of patient 12 (e.g., via spinal cord 16). For example, ultrasound energy 32 may inhibit signals from afferent nerves 20 and/or stretch receptors 22 to the brain of patient 12 that may indicate the need to void bladder 24 to patient 12. In other words, ultrasound modulation of afferent nerves 20 and/or stretch receptors 22 may be selected to reduce signaling that may result in increased detrusor activity.

As another example, ultrasound energy 32 may be configured to modulate nerve tissue including a pudendal nerve 28 that at least partially innervates urinary sphincter 36 of bladder 24 of patient 12. Ultrasound energy 32 may modulate pudendal nerve 28 to stimulate or inhibit constriction and/or relaxation of urinary sphincter 36. In some examples, modulation of pudendal nerve 28 to stimulate or inhibit constriction and/or relaxation of urinary sphincter 36 may help prevent patient 12 from experiencing urinary incontinence.

In other examples, in addition to nerve tissue 26 (e.g., pelvic nerves and/or hypogastric nerves), afferent nerves 20, stretch receptors 22, and/or pudendal nerve 28, ultrasound energy 32 may be configured to additionally, or alternatively, modulate nerve tissue including a sciatic nerve, a tibial nerve, a sacral nerve, a sacral dermatome, a dorsal root ganglion, a dorsal genital nerve, a spinal root, spinal cord 16 within vertebrae 14, or combinations thereof. In this way, modulation of at least one of nerve tissue 26 (e.g., pelvic nerves and/or hypogastric nerves), afferent nerves 20, stretch receptors 22, pudendal nerve 28, a sciatic nerve, a tibial nerve, a sacral nerve, a sacral dermatome, a dorsal root ganglion, a dorsal genital nerve, a spinal root, spinal cord 16, or combinations thereof may result in modulation of the activity of bladder 24, such as bladder activity associated with an overactive bladder, urgency, urinary incontinence, or the like. It is contemplated that wearable ultrasound device 30 may control ultrasound transducers to target ultrasound energy to multiple different nerve tissues simultaneously or at different times (e.g., a time-interleaved manner or as needed according to feedback).

Wearable ultrasound device 30 may be configured to determine a set of parameter values that at least partially defines ultrasound energy 32. Wearable ultrasound device 30 may determine the set of parameter values by accessing the parameter values stored in a memory, based on information from another device, such as an interface device 34, or the like. The set of parameter values defining ultrasound energy 32 may include one or more of an ultrasound frequency, an energy level, a duration, a waveform, an amplitude, an intensity, a set of transducers of wearable ultrasound device 30, or the like. In some examples, the set of parameter values defining ultrasound energy 32 may be based on the desired modulation of the activity of bladder 24. For example, a set of parameter values may define ultrasound energy 32 configured to inhibit one or more stretch receptors 22 or inhibit the activity of nerve tissue 26 (e.g., pelvic nerves and/or hypogastric nerves). Wearable ultrasound device 30 may control delivery of ultrasound energy 32 according to the set of parameter values. Wearable ultrasound device 30 may determine the set of parameter values by retrieving preselected parameter values from memory or selecting parameter values based on sensor and/or user input.

In some examples, the activity of nerve tissue, and therefore bladder activity, may be suppressed or inhibited by slightly heating the neurons of the nerve tissue using ultrasound energy. In some examples, the set of parameter values defining ultrasound energy 32 configured to slightly heat the neurons of the nerve tissue may include a high duty cycle (e.g., a duty cycle greater than about 40%, 50%, 60%, 70%, 80%, or even 90%) of ultrasound energy 32. Such a set of parameter values defining ultrasound energy 32 may result in the neurons of the nerve tissue increasing in temperature by a few degrees Celsius, such as about less than 5° C., less than 3° C. or less than about 2° C. In other examples, the increase in temperature may be less than 2° C. or even less than 1° C. In some examples, wearable ultrasound device 30 may be configured to detect this increase in temperature of target nerve tissue. As an additional example, a set of parameter values configured to suppress and decrease activity of the nerve tissue and/or bladder 24 may define longer bursts of ultrasound energy 32. For example, the set of parameter values may define ultrasound energy 32 with a continuous duration between about 10 milliseconds and about 100 milliseconds, such as, for example, between about 10 milliseconds and about 50 milliseconds or between about 10 milliseconds and about 30 milliseconds. The set of parameter values may also define that ultrasound energy is delivered as a continuous sine waveform during such a duration of time.

As another example, a set of parameter values may be configured to mechanically flex the nerve tissue to increase activity of neurons and bladder 24 or other structures associated with bladder 24. In some such examples, the set of parameter values may define shorter bursts of ultrasound energy 32. For example, the parameters defining such ultrasound energy 32 may include pulses with a duration of between about 1 millisecond and about 10 milliseconds, such as, for example, between about 1 millisecond and about 5 milliseconds or between about 1 millisecond and about 3 milliseconds. In addition, or as an alternative, the set of parameter values configured to mechanically flex and activate nerve tissue may define ultrasound energy 32 with about a relatively smaller duty cycle, such as less than 5% duty cycle or less than 1% duty cycle.

In other examples, the set of parameter values that at least partially defines ultrasound energy may include additional or alternative parameters to define ultrasound energy 32 to activate, suppress, or inhibit nerve tissue, bladder 24, and/or other structures associated with the activity of bladder 24. In some examples, the set of parameter values defines an ultrasound frequency in a range from about 300 kHz to about 2.0 MHz. The ultrasound frequency delivered by the set of ultrasound transducers may be less than an ultrasound frequency typically used for ultrasound imaging. For example, ultrasound frequency used for imaging (e.g., detecting ultrasound energy reflected from anatomical tissues) may use an ultrasound frequency ranging from about 2 MHz to about 10 MHz. The use of lower ultrasound frequencies may reduce the risk of a high temperature increase of the tissues of patient 12 or hearing damage to patient 12. However, higher frequencies may provide higher resolution of imaging in some circumstances. Additionally, or alternatively, the set of parameter values may define ultrasound energy with an energy level of less than about 0.7 watts per square centimeters.

In some examples, wearable ultrasound device 30 may include two or more sets of ultrasound transducers. In some such examples, each set of ultrasound transducers may be configured to deliver ultrasound energy 32 defined by different sets of parameters. For example, a first set of ultrasound transducers may be configured to define ultrasound energy to suppress one or more stretch receptors 22, and a second set of ultrasound transducers may be configured to slightly heat the neurons of nerve tissue 26. In some examples, different sets of ultrasound transducers may be configured to deliver ultrasound energy 32 for different purposes. For example, a first set of ultrasound transducers may be configured to deliver ultrasound energy 32 for therapeutic purposes, and a second set of transducers may be configured to deliver ultrasound energy 32 for imaging purposes. The first set of transducers may include all different transducers than the transducers of the second set of transducers. In this manner, transducers of the first set of transducers may be tuned to a first frequency selected for delivering ultrasound therapy, while transducers of the second set of transducers may be tubed to a second frequency selected for imaging anatomical structures. Wearable ultrasound device 30 may this have different types of ultrasound transducers that are intended to provide respective different functionality. In other examples, however, a same set of ultrasound transducers may be configured to deliver ultrasound energy for both therapeutic purposes and imaging. In some examples, the set of transducers that deliver ultrasound therapy and the set of transducers configured for imaging may include one or more of the same transducers (e.g., one or more transducer may be configured to provide therapy and imaging functionality). In still other examples, three sets of different transducers may be used for therapy and imaging, where a first set of transducers is configured to deliver therapeutic ultrasound energy, a second set of transducers is configured to deliver imaging ultrasound energy, and a third set of transducers may be configured to detect reflected ultrasound waves from the second set of transducers. The ultrasound transducers used for different purposes may be structurally identical, but, in other examples, a first set of ultrasound transducers carried by wearable ultrasound device 30 may be structurally different from a second set of ultrasound transducers carried by wearable ultrasound device 30 for a different purpose. For example, a first set of transducers may be configured to emit ultrasound energy and a second set of transducers may be configured to receive reflected ultrasound energy to image anatomical structures.

Moreover, different sets of ultrasound transducers may be configured to target different locations (e.g., different nerve tissues). For example, a first set of ultrasound transducers may be configured to deliver ultrasound energy 32 that modulates nerve tissue including pudendal nerve 28, and a second set of ultrasound transducers may be configured to deliver ultrasound energy 32 that modulates nerve tissue including one or more stretch receptors 22. In other examples, system 10 may include more than one wearable ultrasound device 30 to target different locations. For example, patient 12 may have a first wearable ultrasound device 30 positioned at a midline of a suprapubic area of patient 12 (e.g., to be positioned approximately near bladder 24) to deliver ultrasound energy 32 to nerve tissue 26, afferent nerves 20, and/or stretch receptors 22, and a second wearable ultrasound device (not shown) positioned on the lower back or upper buttocks area to deliver ultrasound energy 32 to a sacral dermatome and/or a sacral nerve. These different sets of ultrasound transducers may be interspersed about the wearable ultrasound device 30 or positioned in distinct areas of the wearable ultrasound device 30.

In any case, wearable ultrasound device 30 may include a plurality of ultrasound transducers, which may include one or more sets of ultrasound transducers. In some examples, each of the one or more sets of ultrasound transducers may be configured to deliver ultrasound energy 32 defined by different sets of parameters, for different purposes, or the like (e.g., each set of ultrasound transducers is configured a particular way to deliver a particular ultrasound energy 32). In other examples, the one or more sets may be configured to deliver ultrasound energy 32 defined by multiple sets of parameters, for different purposes, or the like (e.g., one or more of the sets of ultrasound transducers can deliver a wider range of ultrasound energy 32). In addition to a set of parameter values defining ultrasound energy 32, the set of parameter values may also identify a set of ultrasound transducers from the plurality of ultrasound transducers to deliver ultrasound energy 32 to the nerve tissue of patient 12. In this way, the plurality of ultrasound transducers may be selectable such that at least some of the plurality of ultrasound transducers including the one or more sets ultrasound transducers are configured to deliver ultrasound energy 32, while a different one or more sets of ultrasound transducers are not configured to deliver ultrasound energy 32 at the same time.

In some examples, a set of ultrasound transducers of wearable ultrasound device 30 may deliver ultrasound energy 32 to patient 12 for imaging. Imaging may be used for visualization of a target region, determining a physiological event, monitoring temperature, monitoring cavitation, evaluating therapy effectiveness, therapy side effects, patient safety, or beam aberration correction. For example, wearable ultrasound device 30 may deliver ultrasound energy 32 to patient 12 for therapeutic purposes, and may image during delivery of such ultrasound energy 32 by detecting reflections of ultrasound energy 32 from one or more tissues of patient 12. In other examples, wearable ultrasound device 30 may image anatomical structures by interleaving delivery of therapeutic ultrasound energy 32 with ultrasound energy 32 intended to elicit reflections detectable by wearable ultrasound device 30. For example, therapeutic ultrasound energy 32 may be of a lower frequency and/or target a different location than ultrasound energy 32 configured for imaging. In this manner, wearable ultrasound device 30 may be configured to deliver therapeutic ultrasound energy and imaging ultrasound energy on a time-interleaved or multiplexed fashion.

In some examples, imaging from wearable ultrasound device 30 may be used to detect a physiological event. For example, wearable ultrasound device 30 may image tissues to detect a size of bladder 24 that may change over time (e.g., increasing size of bladder 24 may indicate a greater likelihood of voiding. Detection of a physiological event may be used to determine one or more parameter values of the set of parameter values. As one example, the size of bladder 24 may be compared to a bladder size threshold, such as a bladder size threshold based on patient history, population data, average bladder sizes, or the like, which may indicate that bladder 24 is full or relatively full (e.g., if the size of bladder 24 exceeds the bladder size threshold). If it is determined that bladder 24 is full or relatively full based on the comparison, one or more parameter values of the set of parameter values may be configured to define ultrasound energy 32 that prevents suppression or inhibition of the nerve tissue, or activates the nerve tissue, such that patient 12 can void bladder 24. For example, as the size of bladder 24 increases, wearable ultrasound device 30 may change the frequency and/or amplitude of ultrasound energy 32 to aid patient 12 in withholding urine in bladder 24. In other examples, the physiological event may include nerve activity, an anxiety level, a patient orientation, a sleep state, a breathing rate, a time of day, an activity level, a blood pressure, electromyography (EMG) information, or a heart rate of patient 12.

Wearable ultrasound device 30 may control delivery of ultrasound energy 32 in response to detecting a physiological event via another sensor or device in addition to, or as an alternative to, wearable ultrasound device 30 detecting the physiological event via imaging or another method utilizing ultrasound energy 32. For example, an accelerometer on wearable ultrasound device 30, elsewhere on patient 12, in a mobile device carried by patient 12 (e.g., interface device 34), or the like may generate a signals representative of a physiological event, such as an increased anxiety level of patient 12. Wearable ultrasound device 30 may interpret fidgeting or movement of patient 12 to be indicative of a symptom from bladder dysfunction, such as an urge to void bladder 24 (e.g., trying to avoid an incontinence event or trying to move to a restroom to void). In turn, wearable ultrasound device 30 may control delivering of ultrasound energy 32 based on the indication of the increased anxiety level. Wearable ultrasound device 30 may incorporate information from other sensors, such as a heart rate sensor, a breathing rate sensor, surface electrodes, or the like, when controlling an ultrasound parameter value and/or delivery of ultrasound energy 32.

In some cases, wearable ultrasound device 30 controls delivery of ultrasound energy 32 in response to detecting the physiological event (e.g., a trigger event). For example, wearable ultrasound device 32 may begin delivering ultrasound energy 32 to the nerve tissue in response to detecting a physiological event, such as delivering ultrasound energy 32 configured to inhibit one or more stretch receptors 22 in response to detecting increased activity of one or more stretch receptors 22 and/or inhibit efferent nerve activity that may signal contractions of the detrusor muscle of bladder 24.

Additionally, or alternatively, system 10 may use input from patient 12 (e.g., via interface device 34) to determine a parameter value of the set of parameter values or control delivery of ultrasound energy 32. In some examples, patient 12 may be able to input information such as an urge to void, an increased urge to void, an involuntary void episode occurred, a void is desired (e.g., system 10 may refrain from providing inhibitory therapy or provide voiding assistive therapy), or the like, to system 10 via interface device 34. System 10 may, responsive to receiving the user input, determine a change to a parameter value of the set of parameter values or otherwise control delivery of ultrasound energy 32. In some cases, patient 12 may be able to modify the therapy delivered by wearable ultrasound device 30 by entering user input (e.g., such as by interface device 34). For example, if patient 12 feels urgency to void bladder 24, patient 12 may be able to control delivery of therapy via interface device 34 such that the activity of bladder 24 and/or nerve tissue associated with bladder 24 is suppressed or inhibited. In this manner, interface device 34 may be configured to receive user input requesting an increase or decrease on the amount of therapy to be delivered or other adjust therapy.

Any combination of information may be used to determine a parameter value of the set of parameter values or control delivery of ultrasound energy 32. For example, system 10 (e.g., interface device 34 and/or wearable ultrasound device 30) may be configured to use any of imaging information, a size of bladder 24, nerve activity, an anxiety level, a patient orientation, a sleep state, a breathing rate, a time of day, an activity level, a blood pressure, EMG information, or a heart rate of patient 12 to determine a parameter value of the set of parameter values or control delivery of ultrasound energy 32. For example, wearable ultrasound device 30 may synthesize information from multiple sensors and identify a change in patient condition, such as the occurrence of a physiological event, when values of multiple signals exceed respective thresholds. Moreover, any of imaging information, a size of bladder 24, nerve activity, an anxiety level, a patient orientation, a sleep state, a breathing rate, a time of day, an activity level, a blood pressure, EMG information, or a heart rate of patient 12 may be detected from one or more devices or sensors, such as wearable ultrasound device 30, interface device 34, input from patient 12, or an accelerometer, a heart rate sensor, a breathing rate sensor, surface electrodes, or the like carried by another device associated with patient 12.

In addition to, or as an alternative to, determining a parameter value of the set of parameter values or controlling delivery of ultrasound energy 32, wearable ultrasound device 30 (or another device of system 10) may control ultrasound delivery using closed loop feedback from sensed patient information such as imaging information, ultrasound energy 32 reflection information, a size of bladder 24, nerve activity, an anxiety level, a patient orientation, a sleep state, a breathing rate, a time of day, an activity level, a blood pressure, EMG information, or a heart rate of patient 12. For example, imaging information and/or ultrasound energy 32 reflection information may be used to map the location of bladder 24 and/or nerve tissue that is to be targeted to receive therapeutic ultrasound energy 32. In turn, such mapping information may be used to select and/or change a set of ultrasound transducers to deliver ultrasound energy or other parameter values that define ultrasound energy 32 such that ultrasound energy 32 reaches the intended target nerve tissue for modulation. The closed loop feedback may also indicate changes in nerve activity or activity of bladder 24, which may indicate that one or more parameter values of a set of parameter values should also be changed.

In some examples, the closed loop feedback may enable system 10 to monitor the therapy delivered by wearable ultrasound device 30. For example, imaging information and/or characteristics of reflected waves from ultrasound energy 32 may be used to a monitor a temperature change, which may be used to monitor ultrasound energy 32 configured to slightly heat neurons of nerve tissue. The closed loop feedback may also provide EMG information, such as EMG amplitude or latency, which may indicate if ultrasound energy 32 is reaching the intended target nerve tissue. In turn, system 10 may be able to determine if ultrasound energy 32 is producing the desired effect, reaching the intended target nerve tissue, or the like, and if one or more parameter values of the set of the parameters should be altered to achieve the desired effect and/or reach the intended target nerve tissue.

System 10 may also be configured to determine intrinsic activity of bladder 24, nerve tissue associated with bladder activity, or some other activity of another anatomical structure. Intrinsic activity may be derived from imaging information, ultrasound energy 32 reflection information, a size of bladder 24, nerve activity, an anxiety level, a patient orientation, a sleep state, a breathing rate, a time of day, an activity level, a blood pressure, EMG information, or a heart rate of patient 12, for example. The intrinsic activity may enable the set of parameters defining ultrasound energy 32 to be determined such that ultrasound energy 32 modulates nerve tissue that at least partially innervates bladder 24 of patient 24 in synchrony with the intrinsic activity. For example, wearable ultrasound device 30 may be configured to detect intrinsic amplitude and/or frequency of nerve impulses and select one or more parameter values of ultrasound energy to amplify and/or inhibit those intrinsic nerve impulses, depending on whether or not nerve inhibiting or activity is desired to treat the bladder activity. In some examples, wearable ultrasound device 30 may determine a pattern (e.g., frequency and/or pulse width) of ultrasound energy to mimic and/or inverse the detected intrinsic patterns of nerve impulses.

In some examples, wearable ultrasound device 30 may also be configured to deliver one or more drugs or other chemicals to patient 12. For example, wearable ultrasound device 30 may be configured to release a drug or other chemical through the skin of patient 12. Such a wearable ultrasound device 30 may be able to deliver drugs or other chemicals to patient 12 at a specific rate from a reservoir carried by wearable ultrasound device 30. The drugs may be passively released or released via an active mechanism such as a drug pump. The drugs or other chemicals released by wearable ultrasound device 30 may be related to the bladder dysfunction of patient 12, or the drugs or other chemicals may be related to something other than the bladder dysfunction of patient 12.

As illustrated in FIG. 1A, system 10 also includes an interface device 34, which may be a computing device having a user interface, e.g., a personal computer, workstation, tablet computing device, or cellular telephone. In some examples, interface device 34 may be a programmer dedicated to controlling the functionality of system 10 as described herein. Interface device 34 may be configured to accept user input from patient 12, such as information relating to the bladder dysfunction (e.g., voiding, urgency, or the like) of patient 12.

In the example of FIG. 1A, interface device 34 is configured to communicate, e.g., via a wireless connection, with wearable ultrasound device 30. In other examples, interface device 34 is configured to communicate, e.g., via a wired connection, with wearable ultrasound device 30. Interface device 34 may also be configured to communicate, e.g., via a wired or wireless connection, with implanted or wearable sensors and/or external sensing devices. Interface device 34 may control, e.g., program, wearable ultrasound device 30, and/or other sensors or sensing devices. Interface device 34 may also receive sensed physiological events from wearable ultrasound device 30, sensors, and/or external sensing devices. Although not illustrated in FIG. 1A, system 10 may include one or more other remote computing devices connected to interface device 34 via a network, and the one or more remote computing devices may control and/or receive information from wearable ultrasound device 30, sensors, and/or external sensing devices via interface device 34. In some examples, interface device 34 and one or more external sensing devices may be integrated as a single device.

System 10 includes one or more processors or other processing circuitry, e.g., of wearable ultrasound device 30, interface device 34, and/or the one or more remote computing devices, that are configured to control wearable ultrasound device 30 and interface device 34, or any other ultrasound device, sensor, sensing device, or any other device described herein to provide the functionality described herein.

For example, the one or more processors or other processing circuitry of one or more of these devices may also be configured to determine a set of parameter values that at least partially defines ultrasound energy 32 configured to modulate nerve tissue that at least partially innervates bladder 24 of patient 12. The one or more processors or other processing circuitry of one or more of these devices may also be configured to control wearable ultrasound device 30 to deliver ultrasound energy 32 from a set of ultrasound transducers external to patient 12 and to the nerve tissue that at least partially innervates bladder 24 of the patient 12. In some examples, the one or more processors or other processing circuitry are configured to detect a physiological event, compare a detected physiological event to a threshold, change a parameter value of the set of parameter values, select a set of ultrasound transducers, image tissue associated with bladder 24, or the like to provide the functionality described herein. Although a single wearable ultrasound device 30 is shown in the example of FIG. 1A, multiple wearable ultrasound devices may be attached to patient 12 and operate independently from each other or communicate with each other to provide coordinated therapy. In one example, one wearable ultrasound device 30 may be configured to deliver ultrasound energy for therapy and another wearable ultrasound device 30 may be configured to generate imaging information using ultrasound.

FIG. 1B is a conceptual and schematic diagram illustrating another example system 40 configured to deliver therapy that treats bladder dysfunction of a patient 12 via a wearable ultrasound device 42. System 40 may be substantially the same as system 10 of FIG. 1A. For example, wearable ultrasound device 42 may be the same or substantially the same as wearable ultrasound device 30 of FIG. 1A. However, as illustrated in FIG. 1B, wearable ultrasound device 42 is configured to communicate with a therapy device 44 via a wired connection, and therapy device 44 is configured to communicate with a programming device 46, e.g., via a wireless or wired connection. In some examples, therapy device 44 and/or programming device 46 may be the same or substantially the same as interface device 34 of FIG. 1A. For example, therapy device 44, programming device 46, or both may be configured to provide the functionality of interface device 34 as described herein.

Therapy device 44 and/or programming device 46 may also be configured to communicate, e.g., via a wired or wireless connection, with implanted or wearable sensors, external sensing devices, or the like. Therapy device 44 may control, e.g., program, wearable ultrasound device 30 and/or other sensors or sensing devices, and programming device 46 may control, e.g., program, therapy device 44 and/or other sensors or sensing devices. Programming device 46 may be a remote computing device, and/or system 40 may include one or more other remote computing devices connected to therapy device 44 or programming device 46 via a network, and the one or more remote computing devices may control and/or receive information from wearable ultrasound device 42, therapy device 44, programming device 46, sensors, and/or external sensing devices. In some examples, therapy device 44 and programming device 46 may be integrated as a single device.

Therapy device 44, and in some examples, programming device 46, may be carried with patient 12 during use of wearable ultrasound device 42. Therapy device 44 and/or programming device 46 may be able to be carried in clothing of patient 12, such as in a pocket, include an adhesive element to attach to a skin surface of patient 12, or may be attached to patient 12 using a strap or other garment. Therapy device 44 may be configured to couple to two or more wearable ultrasound devices 42.

In some examples, system 40 may be used as a trial therapy system. For example, wearable ultrasound device 42 may be larger and include more ultrasound transducers or sensors, which may enable system 40 to determine additional information about patient 12 than wearable ultrasound device 30 of FIG. 1A. In such examples, wearable ultrasound device 42 may include more than 100 ultrasound transducers or even more than 1,000 ultrasound transducers. The large amount of ultrasound transducers may enable system 40 to determine a treatment or diagnostic area of patient 12 (e.g., to position a wearable ultrasound device for chronic use) without being moved during the trial period, determine one or more sets of parameter values to define ultrasound energy 32, determine the efficacy of one or more sets of parameter values defining ultrasound energy 32, or the like. In some examples, patient 12 may wear wearable ultrasound device 42 for a shorter period time, such as less than a month, less than a week, or less than a day, to determine initial information regarding patient 12 and system 40. After wearable ultrasound device 42 indicates therapy efficacy, a clinician may prescribe therapy for patient 12 via wearing wearable ultrasound device 30 for a longer, chronic, period of time. In some examples, wearable ultrasound device 30 may be positioned, controlled, and/or programmed based on at least some of the information determined by wearable ultrasound device 42 during the trial therapy period. In other examples, wearable ultrasound device 30 may function as a trial therapy system, or patient 12 may not use a trial therapy system.

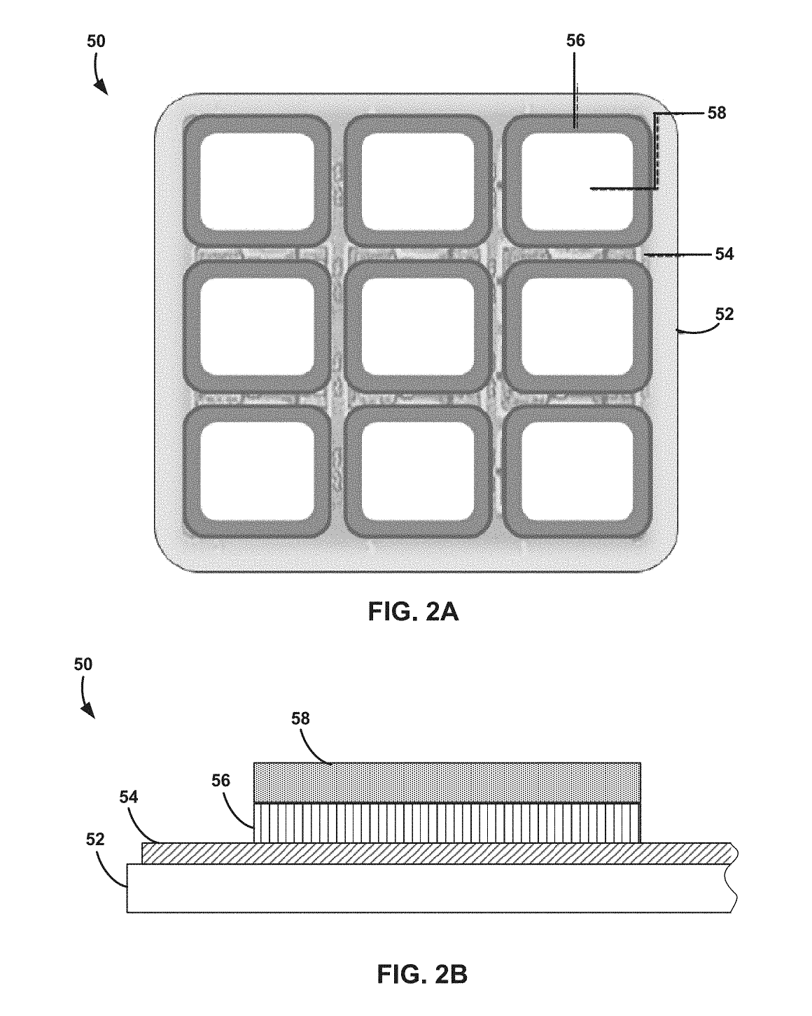

FIGS. 2A and 2B are top-view and side-view diagrams, respectively, illustrating an example wearable ultrasound device 50. In the example of FIGS. 2A and 2B, wearable ultrasound device 50 includes an adhesive layer 52, a flexible interconnect element 54, a plurality of ultrasound transducers 56 connected to flexible interconnect element 54, and a plurality of power sources 58, e.g., batteries, connected to flexible interconnect element 54. FIG. 2B illustrates one ultrasound transducer 56 and one power source 58. Although not illustrated in FIGS. 2A and 2B, wearable ultrasound device 50 may also include signal generation circuitry, one or more processors or processing circuitry, sensing circuitry, and communication circuitry, e.g., configured to communicate with interface device 34 (FIG. 1A) or therapy device 44 (FIG. 1B), connected to flexible interconnect element 54. Wearable ultrasound device 50 may be an example of wearable ultrasound devices 30 or 42 of FIGS. 1A and 1B.

The components of wearable ultrasound device 50 may be configured, e.g., constructed and arranged, such that wearable ultrasound device 50 is flexible. For example, flexible interconnect element 54 may comprise a flexible circuit, e.g., a flex circuit that electrically connects two or more of the components of wearable ultrasound device 50. Flexible interconnect element 54 and adhesive layer 52 may comprise mechanically compliant materials. Additionally, ultrasound transducers 56 and power sources 58 may be discrete and distributed across wearable ultrasound device 50, e.g., in a two-dimensional array as illustrated in FIG. 2A, which may facilitate flexibility of wearable ultrasound device 50. In some examples, signal generation circuitry that drives ultrasound transducers 56 may include flexible driving electronics.

Wearable ultrasound device 50 including plurality of power sources 58 may increase the onboard power capacity of wearable ultrasound device 50. In some examples, power sources 58 include rechargeable batteries. In such examples, wearable ultrasound device 50 may include a recharge interface, such as a coil for inductive recharging or connector, e.g., universal serial bus (USB), mini-USB, or micro-USB, for wired recharging of power sources 58. In some examples, interface device 34 (FIG. 1A), therapy device 44 (FIG. 1B), or another device charges power sources 58 of wearable ultrasound device 50. In other examples, power sources 58 include primary batteries.

In some examples, as illustrated in FIG. 2A, each of power sources 58 is associated with a respective one of ultrasound transducers 56. In some examples, each of power sources 58 is attached to the respective ultrasound transducer 56. In such examples, power sources 58 may be configured as a backing material for ultrasound transducers 56, which may tune an ultrasound frequency of the respective ultrasound transducer 56. Using power sources 58 as a backing material may reduce or eliminate the need for a dedicated backing material to tune ultrasound transducers 56, which may in turn reduce the size, e.g., volume, thickness, or weight, of ultrasound transducers 56. Various features of power sources 58, such as thickness and mass, may be chosen to tune the ultrasound output parameters, e.g., ultrasound frequency. In some examples, flexible interconnect element 54 may also be configured as a backing material for ultrasound transducers 56, in addition to, or instead of, power source 58. In some examples, additional electrical components may be affixed, e.g., directly, to the ultrasound material, e.g., during the manufacturing process, and may act as backing material for ultrasound transducers 56, alone or in combination with other components of wearable ultrasound device 50.

The relative vertical arrangement of adhesive layer 52, interconnect layer 54, ultrasound transducers 56, and power sources 58 illustrated in FIG. 2B is merely one example. In other examples, interconnect layer 54 may be at least partially between ultrasound transducers 56 and power sources 58, or power sources 58 may be at least partially between interconnect layer 54 and ultrasound transducers 56. In some examples, discrete components, such as ultrasound transducers 56 and power sources 58, may be located at least partially within, e.g., may be at least partially surrounded by, interconnect layer 54 and/or adhesive layer 52.

Although nine ultrasound transducers 56 and nine power sources 58 are illustrated in FIG. 2A, in other examples, the numbers of transducers and power sources may be different than illustrated, and/or the number of ultrasound transducers 56 may be different than the number of power sources. In some examples, there may be at least three, at least nine, at least thirty-two, or at least sixty-four ultrasound transducers 56 and/or power sources 58. In some examples, power sources 58 may be horizontally adjacent ultrasound transducers 56. In some examples, one or more power sources 58 may be located anywhere in interconnect element 54 in relation to power transducers (e.g., the signal generation circuitry that drives ultrasound transducers 56) or other components of wearable ultrasound device 50. In other examples, ultrasound device 50 may include hundreds or even thousands of ultrasound transducers 56.

Power sources 58 may be connected in series, in parallel, or in some series/parallel combination. At least partial series combination may boost voltage of the resulting power source 58. To improve acoustic coupling and tune ultrasound transducers 56, the cavity within the power source case (e.g., a battery case) may be substantially free of gas (e.g., free or nearly free), such as by completely filling the space between electrodes with an electrolyte that may be liquid, gel, or solid. In some examples, power sources 58 include battery chemistry that does not generate gas during charge/discharge (for example, using a lithium titanate anode) and/or to allow for removal of gas that is usually formed during the initial charge cycle (known in the art as formation) of the cell. The power source encasement may be a metal such as titanium or aluminum or a metal/polymer foil laminate, although other materials can be used in other examples. The performance of power sources 58 as a backing material may be configured based on acoustic impedance (density×sound speed), thickness, and attenuation coefficient to reduce reflections.

Ultrasound transducers 56 may include a piezoelectric material, such as, lead zirconate titanate (PZT) composite, a PZT film, polyvinylidene fluoride (PVDF), which is a plastic with piezoelectric properties, and/or capacitive micromachined ultrasonic transducers (CMUTs). In other examples, ultrasound transducers 56 may additionally or alternatively include piezoelectric micro machined ultrasonic transducers (PMUTs). PMUTs may draw less power for operation than CMUTs, and, as a result, PMUTs may be advantageous when power is limited. In examples in which power sources 58 and ultrasound transducers 56 are attached, the ultrasound material may be glued or otherwise bonded to the surface of power source 58. In some examples, a metallic housing of power source 58 may be part of an electrical circuit of wearable ultrasound device 50, e.g., to couple ultrasound material of ultrasound transducer 56 to power source 58, signal generation circuitry for driving ultrasound transducer 56, and/or sensing circuitry for processing reflected ultrasound for sensing, diagnostic, or monitoring purposes.

Adhesive layer 52 attaches wearable ultrasound device 50 to patient 12 (FIGS. 1A and 1B). In some examples, adhesive layer 52 is also configured to provide an acoustic interface between ultrasound transducers 56 and tissue of patient 12 for ultrasound. In such examples, the adhesive of adhesive layer 52 may be between, e.g., substantially completely fill the space between, each of ultrasound transducers 56 and an external surface of patient 12.

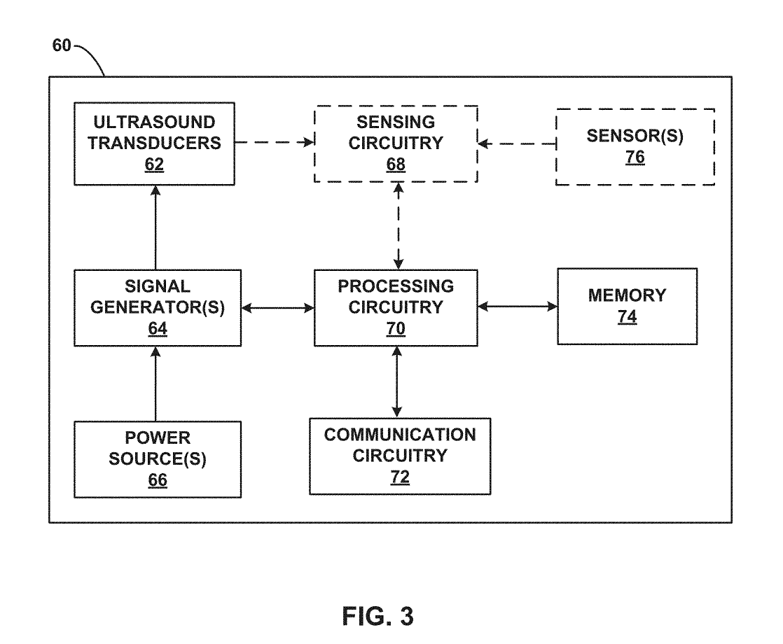

FIG. 3 is a functional block diagram illustrating an example configuration of a wearable ultrasound device 60, which may correspond to any of wearable ultrasound devices 30 (FIG. 1A), 42 (FIG. 1B), or 50 (FIGS. 2A and 2B). As illustrated in FIG. 3, wearable ultrasound device 60 includes one or more processing circuitry 70, a plurality of ultrasound transducers 62, one or more signal generators 64 for driving ultrasound transducers 62 to deliver ultrasound energy, and one or more power sources 66 that provide power to the one or more signal generators 64 for driving ultrasound transducers 62, as well as providing power to other components of wearable ultrasound device 60. Ultrasound transducers 62 and power sources 66 may correspond to any ultrasound transducers, e.g., 56 (FIGS. 2A and 2B), and power sources, e.g., power sources 58 (FIGS. 2A and 2B), respectively, described herein.

As illustrated in FIG. 3, wearable ultrasound device 60 may also include a communication circuitry 72 and memory 74. Memory 74, as well as other memories described herein, may include any volatile or non-volatile media, such as a random access memory (RAM), read only memory (ROM), non-volatile RAM (NVRAM), electrically erasable programmable ROM (EEPROM), flash memory, or the like. Memory 74 may store computer-readable instructions that, when executed by processing circuitry 70, cause wearable ultrasound device 60 to perform various functions described herein. Processing circuitry 70 may include any combination of one or more processors including one or more microprocessors, digital signal processors (DSPs), application specific integrated circuits (ASICs), field programmable gate arrays (FPGAs), or other equivalent integrated or discrete logic circuitry. Accordingly, processing circuitry 70 may include any suitable structure, whether in hardware, software, firmware, or any combination thereof, to perform the functions ascribed herein to processing circuitry 70 and wearable ultrasound device 60.

Processing circuitry 70 are configured to control ultrasound transducers 62 to deliver ultrasound energy, e.g., for a therapeutic or an imaging purpose. More particularly, processing circuitry 70 control signal generators 64 to generate a signal based on power from power sources 66 that drives ultrasound transducers 62 to deliver ultrasound energy. Signal generators 64 may include one or more oscillators configured to generate signals of a desired frequency for the ultrasound energy, amplification or other circuitry to control the amplitude of the driving signals, as well as switching circuitry to selectively direct the signal to one or more of ultrasound transducers 62 and/or selectively control the on/off state of individual ones or sets of transducers 62. Some or all of the signal generation circuitry may be respectively associated with certain sets of ultrasound transducers 62, or shared by all or a subset of ultrasound transducers 62. Processing circuitry 70 may control ultrasound transducers 62 to deliver ultrasound energy to a particular depth, region, or point of tissue, with a particular amplitude, by selecting which of ultrasound transducers 62 is on or driven, and controlling one or more of the amplitude or phase of the driving signal provided to the driven ultrasound transducers 62 by signal generators 64. Different active ultrasound transducers 62 or sets of ultrasound transducers may be driven with different signals, e.g., different amplitudes and/or phases, to target a desired, depth, region, or point of tissue.

In examples in which ultrasound device 60 is configured for imaging or otherwise detecting reflected ultrasound, ultrasound device 60 may include sensing circuitry 68 to selectively, e.g., as controlled by processing circuitry 70, receive and condition electrical signals produced ultrasound transducers 62 as a function of reflected ultrasound, for processing by processing circuitry 70. Sensing circuitry 68 may include one or more switches to control which one or more of transducers 62 are active to sense reflected ultrasound.

In some examples in which wearable ultrasound device 60 is configured to sense physiological events, wearable ultrasound device 60 may include one or more sensors 76, which may correspond to any sensors described herein, such as an accelerometer, a heart rate sensor, a breathing rate sensor, surface electrodes, or the like. Sensing circuitry 68 may selectively, e.g., as controlled by processing circuitry 70, receive and condition electrical signals produced sensors 76 for processing by processing circuitry 70. Sensing circuitry 68 may include one or more switches to control which one or more of sensors are active to sense physiological events.

Power sources 66 may deliver operating power to various components of wearable ultrasound device 60. Power sources 66 may include a small rechargeable or a non-rechargeable battery and a power generation circuit to produce the operating power. Recharging may be accomplished through proximal inductive interaction between a charging device and an inductive charging coil of wearable ultrasound device 60, or a wired connection between the charging device and wearable ultrasound device 60.

Communication circuitry 72 is configured to support wired or wireless communication between wearable ultrasound device 60 and one or more other devices, such as interface device 34, therapy device 44, and/or programming device 46. A user may control the delivery of ultrasound energy by wearable ultrasound device 60, as well as the collection of imaging ultrasound and/or sensing by wearable ultrasound device 60, via communication with processing circuitry 70 through communication circuitry 72. In some examples, programs that control the delivery of ultrasound energy, collection of imaging ultrasound, and/or sensing may be stored in memory 74, and executed by processing circuitry 70. A user may generate or update such programs, using interface device 34, therapy device 44, and/or programming device 46, through communication with wearable ultrasound device 60 via communication circuitry 72. Interface device 34, therapy device 44, programming device 46, and/or another device, may also receive ultrasound images or sensed physiological events collected by processing circuitry 70, or any other information generated by processing circuitry 70, via communication circuitry 72. Such information may be stored in memory 74.

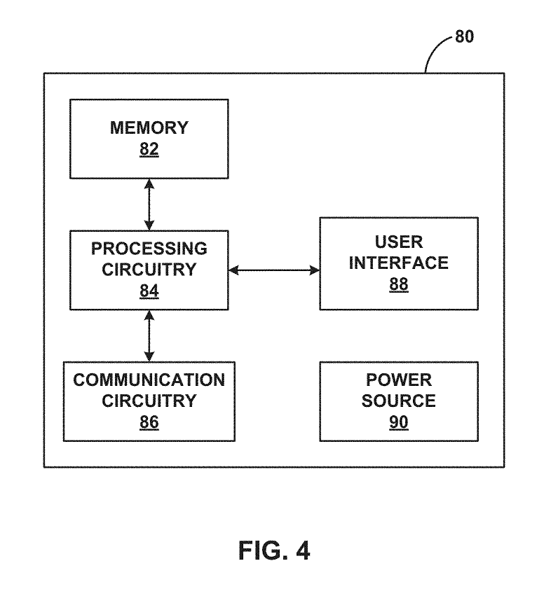

FIG. 4 is a functional block diagram illustrating an example configuration of interface device 80. Interface device 80 may correspond to any of interface device 34 (FIG. 1A), therapy device 44 (FIG. 1B), or programming device 46 (FIG. 2B). As illustrated in FIG. 4, interface device 80 includes processing circuitry 84, a memory 82, a communication circuitry 86, a user interface 88, and a power source 90 configured to power the components of interface device 90. Processing circuitry 84 controls user interface 88 and communication circuitry 86, and stores and retrieves information and instructions to and from memory 82.

Processing circuitry 84 may include any combination of one or more processors including one or more microprocessors, DSPs, ASICs, FPGAs, or other equivalent integrated or discrete logic circuitry. Accordingly, processing circuitry 84 may include any suitable structure, whether in hardware, software, firmware, or any combination thereof, to perform the functions ascribed herein to processing circuitry 84 and interface device 80. Memory 82 may include program instructions that, when executed by processing circuitry 84, cause processing circuitry 84 and interface device 80 to perform any of the functions ascribed to them herein. Memory 82 may include any volatile or nonvolatile memory, such as RAM, ROM, EEPROM or flash memory.

A user, such as a clinician, other caregiver, or patient 12 (FIGS. 1A and 1B), may interact with interface device 90 through user interface 88. User interface 88 includes a display, with which processing circuitry 84 may present information, such as information relating to bladder or nerve tissue activity, or other information retrieved from wearable ultrasound device 60 (FIG. 3). In addition, user interface 88 may include an input mechanism to receive input from the user, though which the user may control or program delivery of ultrasound energy or input occurrence of a physiological event according to any of the techniques described herein. Communication circuitry 86 is configured for wired or wireless communication with the corresponding communication circuitry 64 of ultrasound device 60 (FIG. 3), to facilitate user control or programming of wearable ultrasound device 60, or retrieval of information from wearable ultrasound device 60.

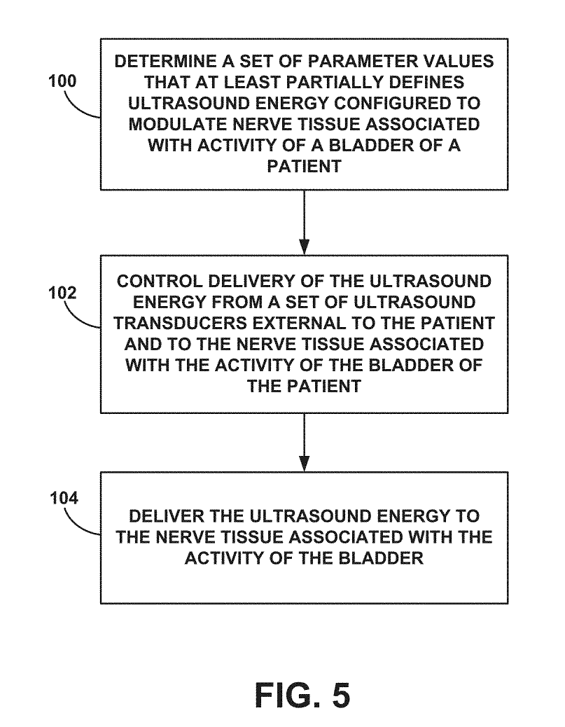

FIG. 5 is a flow diagram illustrating an example technique of modulating bladder activity of a patient via a wearable ultrasound device. The technique of FIG. 5 will be described with respect to system 10 of FIG. 1A. In other examples, however, the technique of FIG. 5 may be used with a system other than system 10 of FIG. 1A, such as with example system 40 of FIG. 1B.

The technique of FIG. 5 includes processing circuitry of wearable ultrasound device 30 determining a set of parameter values that at least partially defines ultrasound energy 32 configured to modulate nerve tissue (e.g., nerve tissue 26, pudendal nerve 28, afferent nerves 20, and/or stretch receptors 22) associated with activity of bladder 24 of patient 12 (100). For example, the nerve tissue may at least partially innervate bladder 24 or another structure (e.g., a sphincter) associated with the activity of bladder 24. Determining the set of parameter values may include accessing the parameter values stored in a memory of wearable ultrasound device 30, determining the set of parameter values based on information from another device, such as an interface device 34, or determining the set of parameter values for ultrasound energy based on one or more sensors that generate information representative of the physiological condition of patient 12. The set of parameter values defining ultrasound energy 32 may include one or more of an ultrasound frequency, an energy level, a duration, a waveform, an amplitude, an intensity, a set of transducers of wearable ultrasound device 30, or other parameter values that define ultrasound energy 32. In some examples, the set of parameter values defining ultrasound energy 32 may be based on the desired modulation of the activity of bladder 24. For example, a set of parameter values may define ultrasound energy 32 configured to inhibit one or more stretch receptors 22 or inhibit the activity of nerve tissue 26 (e.g., pelvic nerves and/or hypogastric nerves). In other examples, the set of parameters may define ultrasound energy 32 configured to inhibit, suppress, and/or activate one or more additional or alternative nerve tissues configured to innervate bladder 24.

In some examples, determining the set of parameter values may include determining one or more parameter values of the set of parameter values based on detecting a physiological event. For example, detecting the physiological event may include detecting the size of bladder 24. The size of bladder 24 may be compared to a bladder size threshold, such as a bladder size threshold based on patient history, data from the general population, average bladder sizes, or the like, which may indicate that bladder 24 is full or relatively full (e.g., if the size of bladder 24 exceeds the bladder size threshold) or otherwise that therapeutic ultrasound energy should be delivered to intervene in bladder activity. If wearable ultrasound device 30 determines that bladder 24 is full or relatively full based on the comparison, one or more parameter values of the set of parameter values may be configured to define ultrasound energy 32 that prevents suppression or inhibition of the nerve tissue, or activates the nerve tissue, such that patient 12 can void bladder 24. In this manner, wearable ultrasound device 30 may initiate delivery of ultrasound energy in response to detecting a physiological event.

In some examples, wearable ultrasound device 30 may detect the physiological event, such as via imaging. In other examples, a sensor on wearable ultrasound device 30 or another sensor (e.g., an accelerometer, one or more electrodes, a temperature sensor, etc.) may generate signals used by wearable ultrasound device 30 to detect the physiological event. Moreover, in some examples, more than one physiological event may be used to determine one or more parameter values of the set of parameter values.

The technique of FIG. 5 further includes controlling, according to the set of parameter values, delivery of ultrasound energy 32 from a set of ultrasound transducers external to patient 12 and to the nerve tissue associated with activity of bladder 24 of patient 12 (102). For example, wearable ultrasound device 30 may control delivery of ultrasound energy 32 to begin, stop, or continue delivery of the ultrasound energy. Wearable ultrasound device 30 may also change one or more parameter values that define ultrasound energy 34 to control delivery of ultrasound energy 32.

In some cases, wearable ultrasound device 30 may control delivery of ultrasound energy 32 in response to detecting a physiological event. For example, wearable ultrasound device 30 may determine that a signal value representative of a physiological condition exceeds a threshold to identified that the physiological event has occurred. In response to detecting this physiological event, wearable ultrasound device 30 may control delivery of ultrasound energy 32 may to begin delivery of ultrasound energy 32. Examples of a physiological event may include detection of increased activity of one or more stretch receptors 22, increased movement or fidgeting of the patient, or other physiological events that may be related to the activity of bladder 24 and/or bladder dysfunction. Additionally, or alternatively, controlling delivery of ultrasound energy 32 may be in response to user input from patient 12, such as through the use of interface device 34.

The technique of FIG. 5 also includes delivering ultrasound energy 32 to the nerve tissue that at least partially innervates bladder 24 (104). Ultrasound energy 32 may be delivered via one or more sets of ultrasound transducers of wearable ultrasound device 30. In some examples, delivering ultrasound energy 32 includes delivering ultrasound energy 32 with an ultrasound frequency in a range from about 500 kHz to about 1.5 MHz and/or with an energy level of less than about 0.7 watts per square centimeters, as may be defined by the set of parameter values determined in step 100.

In some examples, delivering ultrasound energy 32 may include slightly heating the neurons of the nerve tissue, such as increasing the temperature of the nerve tissue by about less than 5° C., less than about 3° C., or by less than about 2° C. In other examples, delivering ultrasound energy 32 may include delivering longer bursts of ultrasound energy 32. For example, delivering burst of ultrasound energy 32 for a duration between about 10 milliseconds and about 100 milliseconds, such as, for example, between about 10 milliseconds and about 50 milliseconds or between about 10 milliseconds and about 30 milliseconds. As another example, delivering ultrasound energy 32 may include mechanically flexing the nerve tissue to increase activity of the nerve tissue and/or bladder 24. In some such examples, delivering ultrasound energy 32 may include delivering shorter bursts of ultrasound energy 32, such as delivering ultrasound energy 32 in pulses with a duration of between about 1 millisecond and about 10 milliseconds, such as, for example, between about 1 millisecond and about 5 milliseconds or between about 1 millisecond and about 3 milliseconds.

The technique of FIG. 5 to modulate nerve tissue configured to at least partially innervate bladder 24 of patient 12 may be used for a therapeutic purpose to manage, alleviate, control, or reduce bladder dysfunction and/or symptoms associated with bladder dysfunction. In some examples, wearable ultrasound device 30 may additionally, or alternatively, be configured to detect reflected ultrasound waves for imaging physiological structures which may include generating information related to structure dimensions and distances between structures. This imaging information may be used to, for example, target ultrasound energy to appropriate never tissue and/or monitor activity of the bladder or other structures associated with the activity of bladder 24.

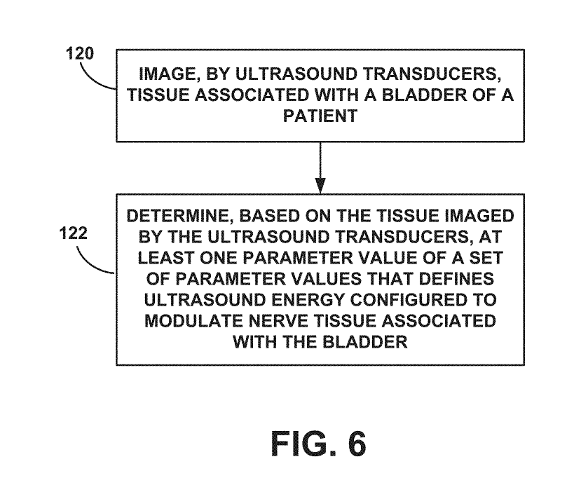

FIG. 6 is a flow diagram illustrating an example technique of determining a set of parameters based on imaging via a wearable ultrasound device. The technique of FIG. 6 will be described with respect to system 10 of FIG. 1A. In other examples, however, the technique of FIG. 6 may be used with a system other than system 10 of FIG. 1A, such as with system 40 of FIG. 1B.

The technique of FIG. 6 includes imaging tissue associated with bladder 24 (120). In some examples, imaging tissue associated with bladder 24 includes delivering ultrasound energy 32 to the tissue associated with bladder 24 and receiving reflected ultrasound energy (e.g., mechanical waves resulting from initial ultrasound waves bouncing off tissues in patient 12) from tissue associated with bladder 24 and/or other tissues of patient 12. In some cases, imaging tissue associated with bladder 24 may be performed during delivery of ultrasound energy 32 for therapeutic purposes (e.g., monitoring patient activity or changes to anatomical structures that may inform changes to one or more ultrasound parameter values). In other cases, imaging tissue associated with bladder 24 may include delivering ultrasound energy 32 configured of a frequency and/or intensity specific for imaging. For example, prior to delivering therapeutic ultrasound energy 32, a burst of imaging ultrasound energy 32 may be delivered to patient 12, and reflected ultrasound waves may be detected to generate information about the current physiological or anatomical state of the patient.

In some examples, imaging tissue associated with bladder 24 may be used to detect a physiological event, such as a size of bladder 24, nerve activity, an anxiety level, a patient orientation, a sleep state, a breathing rate, a time of day, an activity level, a blood pressure, EMG information, or a heart rate of patient 12. The wearable ultrasound device 30 may, in some examples, contextualize the imaging information with other physiological information detected from other sensors (non-ultrasound sensors) associated with patient 12.

The technique of FIG. 6 further includes determining at least one parameter value of a set of parameter values based on the image of the tissue associated with bladder 24 (122). In some examples, imaging including detection of a physiological event may be used to determine a parameter value of the set of parameter values that define ultrasound energy 24. For example, imaging associated with the activity of bladder 24 or tissue associated with bladder 24 may be used to detect the size of bladder 24, which, in turn may be used to determine a parameter value of the set of parameter values defining ultrasound energy 32. In other examples, the image of tissue associated with bladder 24 may be used in a manner other than to detect a physiological event to determine at least one parameter value of a set of parameter values based on the image of the tissue associated with bladder 24. As one example, the image of tissue associated with bladder 24 may be used to map the location of bladder 24 and/or nerve tissue. In turn, such mapping information may be used to determine a parameter value such as a selection of a set of ultrasound transducers. For example, wearable ultrasound device 30 may use ultrasound imaging to identify nerve activity associated with the bladder dysfunction and then target ultrasound energy to that nerve tissue of the activity in order to modulate the bladder activity.

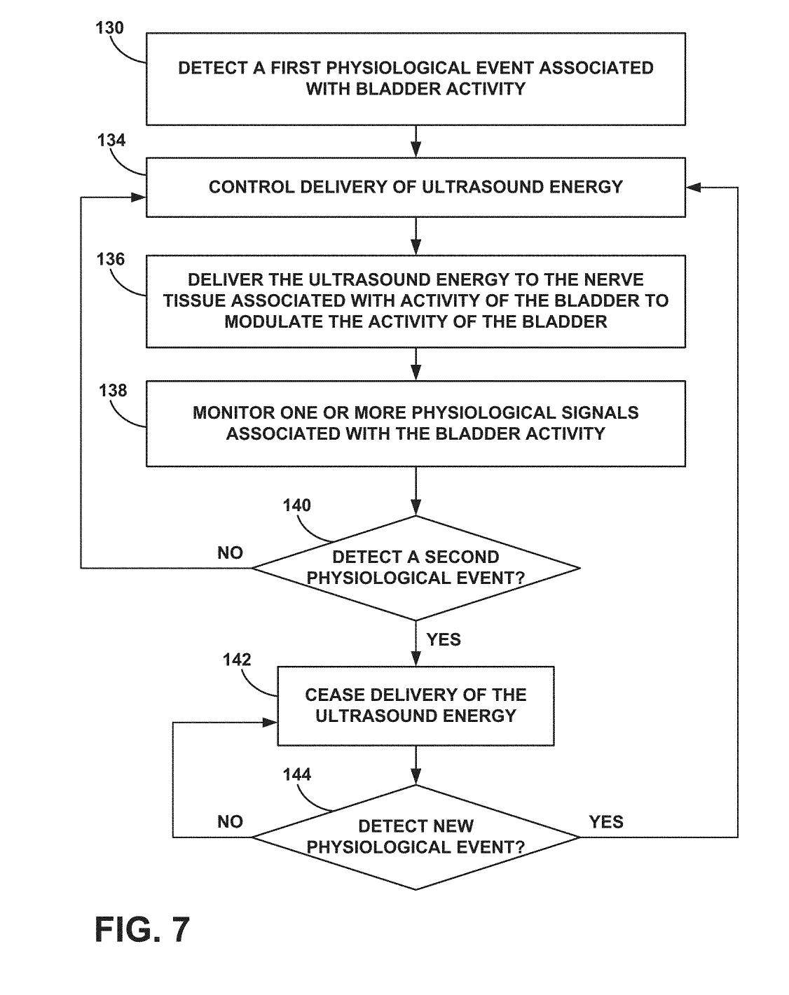

FIG. 7 is a flow diagram illustrating an example technique of delivering ultrasound energy to nerve tissue of a patient based on detection of a physiological event, such as a trigger event. The example technique of FIG. 7 will be described with respect to system 10 of FIG. 1A. In other examples, however, the technique of FIG. 7 may be used with a system other than system 10 of FIG. 1A, such as with system 40 of FIG. 1B.

The technique of FIG. 7 includes detecting a first physiological event associated with bladder activity (130). In some examples, detecting the first physiological event associated with bladder activity may include one or more of detecting a size of bladder 24, nerve activity associated with bladder 24, an anxiety level, a patient orientation, a sleep state, a breathing rate, a time of day, an activity level, a blood pressure, EMG information, or a heart rate of patient 12. For example, an increased anxiety level or change in patient orientation (e.g., standing from a lying down position) may indicate that the patient 12 is expecting an upcoming incontinence event. The first physiological event may be detected via imaging by wearable ultrasound device 30, one or more non-ultrasound sensors carried by wearable ultrasound device 30, one or more sensors attached to or implanted within patient 12, and/or by one or more external devices, such as interface device 34.

Wearable ultrasound device 30 may also determine a set of parameters that at least partially defines ultrasound energy 32 configured to modulate nerve tissue that at least partially innervates bladder 24 of patient 12. Determining the set of parameters may be based on the detected first physiological event in some cases. In other examples, wearable ultrasound device 30 may refer to stored parameter values preselected for delivery of ultrasound energy. In response to detecting the first physiological event (130), wearable ultrasound device 30 controls delivery of ultrasound energy 32 to the nerve tissue associated with activity of the bladder 24 to modulate activity of the bladder (136).

The technique of FIG. 7 additionally includes monitoring one or more physiological events associated with bladder 24 (138) and detecting a second physiological event associated with bladder activity (140) that may indicate ultrasound energy may be stopped. For example, wearable ultrasound device 30 may be configured to image one or more structures of patient 12, receive information from one or more sensors of wearable ultrasound device 30, receive information from one or more sensors externally worn by or implanted within patient 12, and/or one or more external devices, such as interface device 34 to monitor one or more physiological events. Example physiological events may include a size of bladder 24, nerve activity associated with bladder 24, an anxiety level, a patient orientation, a sleep state, a breathing rate, a time of day, an activity level, a blood pressure, EMG information, a heart rate of patient 12, a temperature of tissue of patient 12, or the like. In some examples, monitoring such physiological events and detecting the second physiological event associated with activity of bladder 24 may enable system 10 to utilize closed loop feedback.

If wearable ultrasound device 30 does not detect a second physiological event (“NO” branch of block 140), wearable ultrasound device 30 may continue to control delivery of ultrasound energy (134). During delivery of ultrasound energy, wearable ultrasound device 30 may monitor other physiological information to detect one or more additional events that may trigger wearable ultrasound device 30 to change one or more ultrasound parameter values to accommodate changing physiology, such as changes in patient orientation (e.g., posture), changes to bladder volume, detecting of stretch receptor activity, or any other events related to bladder activity. As one example, detection of another physiological event may indicate ultrasound energy 32 is not reaching the intended target, is not efficacious, or the like. As one example, imaging information and/or ultrasound energy 32 reflection information may be monitored to determine if ultrasound energy 32 is having a desired effect, such as, increasing the temperature of nerve tissue associated with bladder 24, inhibiting stretch receptors 22, or other desired effects from the delivery of ultrasound energy 32. In response to wearable ultrasound device 30 determining that ultrasound therapy is not effective, wearable ultrasound device 30 may adjust one or more ultrasound parameter values (e.g., change ultrasound intensity, ultrasound frequency, targeted location, or the ultrasound transducers used to deliver ultrasound energy).

In some examples, the one or more physiological events associated with bladder 24 may be monitored for a predetermined amount of time. For example, the one or more physiological events may be monitored for a predetermined amount of time after delivery of ultrasound energy 32 (136).

If wearable ultrasound device 30 detects a second physiological event (“YES” branch of block 140), wearable ultrasound device 30 ceases delivery of ultrasound energy 32. For example, the physiological event may be a determined voiding event by patient 12. The second physiological event may be indicated via a user input to interface device 32 representative of a desired voiding event. Alternatively, wearable ultrasound device 30 may use an accelerometer and/or EMG signals to determine that patient 12 is attempting to sleep, and wearable ultrasound device 30 may be configured to turn off ultrasound therapy under these conditions. In other examples, wearable ultrasound device 30 may cease ultrasound delivery after a predetermined timer elapses or according to a certain time of day. After ultrasound energy has been stopped (142), wearable ultrasound device 30 may then determine if a new physiological event (e.g., the first physiological event or another event) is detected (144). If no new physiological event is detected (“NO” branch of block 144), wearable ultrasound device 30 may continue to refrain from delivering ultrasound energy (142). If wearable ultrasound device 30 detects the new physiological event (“YES” branch of block 144), wearable ultrasound device 30 may again control delivery of ultrasound energy (143).

Although FIG. 7 is described with respect to detecting a physiological event and controlling delivery of ultrasound therapy based on the detected physiological event, other events may be used by system 10 to control ultrasound delivery. For example, wearable ultrasound device 30 may be configured to detect two or more different physiological events (e.g., events of different physiological aspects from a single sensor or events from different sensors) before beginning to deliver ultrasound energy). In addition, or alternatively, wearable ultrasound device 30 may begin or stop delivering ultrasound energy in response to a user input based trigger event. For example, interface device 32 may receive user input requesting therapy to avoid an urge incontinence event, and wearable ultrasound device 30 may begin delivering ultrasound therapy in response to receiving an indication of the received user input.

The following examples are examples described herein.

### Example 1

A method for modulating bladder activity, the method comprising: determining, by processing circuitry, a set of parameter values that at least partially defines ultrasound energy configured to modulate nerve tissue associated with activity of a bladder of a patient; and controlling, by processing circuitry and according to the set of parameter values, delivery of the ultrasound energy from a set of ultrasound transducers external to the patient and to the nerve tissue associated with the activity of the bladder of the patient.

### Example 2

The method of example 1, further comprising delivering, by the set of ultrasound transducers, the ultrasound energy to the nerve tissue associated with the activity of the bladder.

### Example 3

The method of any of examples 1 and 2, wherein the nerve tissue comprises one or more stretch receptors associated with the bladder, and wherein determining the set of parameter values comprises determining the set of parameter values that at least partially defines ultrasound energy configured to inhibit the one or more stretch receptors.

### Example 4

The method of any of examples 1 through 3, wherein the nerve tissue comprises at least one of a pudendal nerve, a pelvic nerve, a hypogastric nerve, an afferent nerve, a sciatic nerve, a tibial nerve, a sacral nerve, a sacral dermatome, a dorsal root ganglion, a spinal root, or a spinal cord.

### Example 5

The method of any of examples 1 through 4, wherein the set of parameter values comprises an ultrasound frequency in a range from 300 kHz to 2.0 MHz.

### Example 6

The method of any of examples 1 through 5, wherein the set of parameter values comprises an energy level of less than 0.7 watts per square centimeters.

### Example 7

The method of any of examples 1 through 6, further comprising detecting a physiological event associated with bladder activity, and wherein controlling delivery of the ultrasound energy comprises, responsive to detecting the physiological event, controlling the set of ultrasound transducers to begin delivering the ultrasound energy to the nerve tissue.

### Example 8

The method of example 7, wherein a plurality of ultrasound transducers comprises the set of ultrasound transducers, and wherein detecting the physiological event comprises detecting, via at least one ultrasound transducer of the plurality of ultrasound transducers, a size of the bladder exceeding a bladder size threshold.

### Example 9

The method of any of examples 7 and 8, wherein detecting the physiological event comprises detecting, via a sensor, at least one of an anxiety level, a patient orientation, a sleep state, a breathing rate, electromyography (EMG) information, or a heart rate.

### Example 10

The method of any of examples 1 through 9, wherein a plurality of ultrasound transducers comprises the set of ultrasound transducers, and wherein the method further comprises imaging, with at least one ultrasound transducer of the plurality of ultrasound transducers, tissue associated with the bladder, and wherein determining the set of parameter values comprises determining, based on the imaging of the tissue associated with the bladder, at least one parameter value of the set of parameter values.

### Example 11

The method of example 10, wherein the at least one parameter value identifies, from a plurality of selectable ultrasound transducers, the set of ultrasound transducers for delivering the ultrasound energy to the nerve tissue.

### Example 12

The method of any of examples 1 through 11, wherein an external patch comprises the set of ultrasound transducers and the processing circuitry, and wherein the external patch is configured to be attached to an external skin surface of the patient.

### Example 13

A system for modulating bladder activity, the system comprising: processing circuitry configured to: determine a set of parameter values that at least partially defines ultrasound energy configured to modulate nerve tissue associated with activity of a bladder of a patient; and control, according to the set of parameter values, delivery of the ultrasound energy from a set of ultrasound transducers external to the patient and to the nerve tissue associated with the activity of the bladder of the patient.

### Example 14

The system of example 13, further comprising the set of ultrasound transducers configured to deliver the ultrasound energy to the nerve tissue associated with the activity of the bladder.

### Example 15

The system of any of examples 13 and 14, wherein the nerve tissue comprises one or more stretch receptors associated with the bladder, and wherein the processing circuitry is configured to determine the set of parameter values that at least partially defines ultrasound energy configured to inhibit the one or more stretch receptors.

### Example 16

The system of any of examples 14 through 15, wherein the nerve tissue comprises at least one of a pudendal nerve, a pelvic nerve, a hypogastric nerve, an afferent nerve, a sciatic nerve, a tibial nerve, a sacral nerve, a sacral dermatome, a dorsal root ganglion, a spinal root, or a spinal cord.

### Example 17

The system of any of examples 13 through 16, wherein the set of parameter values comprises an ultrasound frequency in a range from 300 kHz to 2.0 MHz.

### Example 18

The system of any of examples 13 through 17, wherein the processing circuitry is configured to: detect a physiological event associated with bladder activity; and responsive to detecting the physiological event, control the set of ultrasound transducers to begin delivering the ultrasound energy to the nerve tissue.

### Example 19

The system of example 18, wherein a plurality of ultrasound transducers comprises the set of ultrasound transducers, and wherein the processing circuitry is configured to detect, via at least one ultrasound transducer of the plurality of ultrasound transducers, a size of the bladder exceeding a bladder size threshold.

### Example 20

The system of any of examples 18 and 19, wherein the processing circuitry is configured to detect, via a sensor, at least one of an anxiety level, a patient orientation, a sleep state, a breathing rate, electromyography (EMG) information, or a heart rate.

### Example 21

The system of any of examples 13 through 20, wherein a plurality of ultrasound transducers comprises the set of ultrasound transducers, and wherein the processing circuitry is further configured to: image, with at least one ultrasound transducer of the plurality of ultrasound transducers, tissue associated with the bladder; and determine, based on the imaging of the tissue associated with the bladder, at least one parameter value of the set of parameter values.

### Example 22

The system of any of examples 13 through 21, further comprising an external patch comprising the set of ultrasound transducers and the processing circuitry, and wherein the external patch is configured to be attached to an external skin surface of the patient.

### Example 23

A non-transitory computer readable medium comprising instructions that, when executed, cause processing circuitry to: determine a set of parameter values that at least partially defines ultrasound energy configured to modulate nerve tissue associated with activity of a bladder of a patient; and control, according to the set of parameter values, delivery of the ultrasound energy from a set of ultrasound transducers external to the patient and to the nerve tissue associated with the activity of the bladder of the patient.

### Example 24

A system for modulating bladder activity, the system comprising: means for determining a set of parameter values that at least partially defines ultrasound energy configured to modulate nerve tissue associated with activity of a bladder of a patient; and means for controlling, according to the set of parameter values, delivery of the ultrasound energy from a set of ultrasound transducers external to the patient and to the nerve tissue associated with the activity of the bladder of the patient.

### Example 25

The system of example 24, further comprising the set of ultrasound transducers configured to deliver the ultrasound energy.

The techniques described in this disclosure, including those attributed to system 10, system 40, wearable ultrasound device 30, wearable ultrasound device 42, and wearable ultrasound device 60, and various constituent components, may be implemented, at least in part, in hardware, software, firmware or any combination thereof. For example, various aspects of the techniques may be implemented within one or more processors or processing circuitry, including one or more microprocessors, DSPs, ASICs, FPGAs, or any other equivalent integrated or discrete logic circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques or processes described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

What is claimed is:

1. A method for modulating bladder activity, the method comprising:
determining, by processing circuitry, a set of parameter values that at least partially defines ultrasound energy configured to modulate nerve tissue associated with activity of a bladder of a patient; and
controlling, by processing circuitry and according to the set of parameter values, delivery of the ultrasound energy from a set of ultrasound transducers external to the patient and to the nerve tissue associated with the activity of the bladder of the patient.

2. The method of claim 1, further comprising delivering, by the set of ultrasound transducers, the ultrasound energy to the nerve tissue associated with the activity of the bladder.

3. The method of claim 1, wherein the nerve tissue comprises one or more stretch receptors associated with the bladder, and wherein determining the set of parameter values comprises determining the set of parameter values that at least partially defines ultrasound energy configured to inhibit the one or more stretch receptors.

4. The method of claim 1, wherein the nerve tissue comprises at least one of a pudendal nerve, a pelvic nerve, a hypogastric nerve, an afferent nerve, a sciatic nerve, a tibial nerve, a sacral nerve, a sacral dermatome, a dorsal root ganglion, a spinal root, or a spinal cord.

5. The method of claim 1, wherein the set of parameter values comprises an ultrasound frequency in a range from 300 kHz to 2.0 MHz.

6. The method of claim 1, wherein the set of parameter values comprises an energy level of less than 0.7 watts per square centimeters.

7. The method of claim 1, further comprising detecting a physiological event associated with bladder activity, and wherein controlling delivery of the ultrasound energy comprises, responsive to detecting the physiological event, controlling the set of ultrasound transducers to begin delivering the ultrasound energy to the nerve tissue.

8. The method of claim 7, wherein a plurality of ultrasound transducers comprises the set of ultrasound transducers, and wherein detecting the physiological event comprises detecting, via at least one ultrasound transducer of the plurality of ultrasound transducers, a size of the bladder exceeding a bladder size threshold.

9. The method of claim 7, wherein detecting the physiological event comprises detecting, via a sensor, at least one of an anxiety level, a patient orientation, a sleep state, a breathing rate, electromyography (EMG) information, or a heart rate.

10. The method of claim 1, wherein a plurality of ultrasound transducers comprises the set of ultrasound transducers, and wherein the method further comprises imaging, with at least one ultrasound transducer of the plurality of ultrasound transducers, tissue associated with the bladder, and wherein determining the set of parameter values comprises determining, based on the imaging of the tissue associated with the bladder, at least one parameter value of the set of parameter values.

11. The method of claim 10, wherein the at least one parameter value identifies, from a plurality of selectable ultrasound transducers, the set of ultrasound transducers for delivering the ultrasound energy to the nerve tissue.

12. The method of claim 1, wherein an external patch comprises the set of ultrasound transducers and the processing circuitry, and wherein the external patch is configured to be attached to an external skin surface of the patient.

13. A system for modulating bladder activity, the system comprising:
processing circuitry configured to:
determine a set of parameter values that at least partially defines ultrasound energy configured to modulate nerve tissue associated with activity of a bladder of a patient; and
control, according to the set of parameter values, delivery of the ultrasound energy from a set of ultrasound transducers external to the patient and to the nerve tissue associated with the activity of the bladder of the patient.

14. The system of claim 13, further comprising the set of ultrasound transducers configured to deliver the ultrasound energy to the nerve tissue associated with the activity of the bladder.

15. The system of claim 13, wherein the nerve tissue comprises one or more stretch receptors associated with the bladder, and wherein the processing circuitry is configured to determine the set of parameter values that at least partially defines ultrasound energy configured to inhibit the one or more stretch receptors.

16. The system of claim 13, wherein the nerve tissue comprises at least one of a pudendal nerve, a pelvic nerve, a hypogastric nerve, an afferent nerve, a sciatic nerve, a tibial nerve, a sacral nerve, a sacral dermatome, a dorsal root ganglion, a spinal root, or a spinal cord.

17. The system of claim 13, wherein the set of parameter values comprises an ultrasound frequency in a range from 300 kHz to 2.0 MHz.

18. The system of claim 13, wherein the processing circuitry is configured to:
detect a physiological event associated with bladder activity; and
responsive to detecting the physiological event, control the set of ultrasound transducers to begin delivering the ultrasound energy to the nerve tissue.

19. The system of claim 18, wherein a plurality of ultrasound transducers comprises the set of ultrasound transducers, and wherein the processing circuitry is configured to detect, via at least one ultrasound transducer of the plurality of ultrasound transducers, a size of the bladder exceeding a bladder size threshold.

20. The system of claim 18, wherein the processing circuitry is configured to detect, via a sensor, at least one of an anxiety level, a patient orientation, a sleep state, a breathing rate, electromyography (EMG) information, or a heart rate.

21. The system of claim 13, wherein a plurality of ultrasound transducers comprises the set of ultrasound transducers, and wherein the processing circuitry is further configured to:
image, with at least one ultrasound transducer of the plurality of ultrasound transducers, tissue associated with the bladder; and
determine, based on the imaging of the tissue associated with the bladder, at least one parameter value of the set of parameter values.

22. The system of claim 13, further comprising an external patch comprising the set of ultrasound transducers and the processing circuitry, and wherein the external patch is configured to be attached to an external skin surface of the patient.

23. A non-transitory computer readable medium comprising instructions that, when executed, cause processing circuitry to:
determine a set of parameter values that at least partially defines ultrasound energy configured to modulate nerve tissue associated with activity of a bladder of a patient; and
control, according to the set of parameter values, delivery of the ultrasound energy from a set of ultrasound transducers external to the patient and to the nerve tissue associated with the activity of the bladder of the patient.
